(19) United States
(12) Patent Application Publication
(10) Pub. No.: US 2007/0128727 A1
(43) Pub. Date: Jun. 7, 2007

(54) Methods for differentiation of embryonic stem cells

(76) Inventors: Fredric B. Kraemer, Palo Alto, CA (US); Wen-Jun Shen, Palo Alto, CA (US); Theresa L. Chen, Palo Alto, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/593,667

(22) Filed: Nov. 6, 2006

Related U.S. Application Data
(60) Provisional application No. 60/734,889, filed on Nov. 8, 2005.

(51) Int. Cl. C12N 15/09; C12N 5/08

(52) U.S. Cl. 435/455; 435/366

(57) ABSTRACT

Methods are provided for the in vitro differentiation of one or both of adipocytes and osteoblasts from embryonic stem cells.

### BACKGROUND OF THE INVENTION

The growth potential of mammalian embryonic stage cells have been known for many years, but the ability to culture such pluripotent and totipotent stem cells, particularly human stem cells, has only been recently developed. Stem cells have a capacity both for self-renewal and the generation of differentiated cell types. Embryonic stem (ES) cells are derived from cultures of inner cell mass (ICM) cells, and have the property of participating as totipotent cells when placed into host blastocysts. The developmental pathways that endogenous ICM cells or transferred ES cells take to tissue formation and organogenesis has led many to hope that these pathways can be controlled for the development of tissue and organ specific stem cells.

ES cells can generally be maintained in an undifferentiated state indefinitely without losing differentiation potential. In contrast, adult stem cells usually proliferate for a limited number of generations. The differentiation of adult stem cells is also usually restricted to certain tissue types (multipotent). In contrast, ES cells are generally regarded as pluripotent and can develop into multiple tissue types, for example when injected into a host.

Stem cell fate is controlled by both intrinsic regulators and the extracellular environment (niche). Under certain conditions in cell culture, stem cells can differentiate spontaneously. For example, when ES cells are grown in suspension in the absence of leukemia inhibitory factors, they form aggregates called embryoid bodies, which begin to differentiate spontaneously into various cell types, including hematopoietic, endothelial, neuronal and muscle cells. However, spontaneous differentiation is generally inefficient and leads to heterogeneous populations of differentiated and undifferentiated cells, which are not useful for cell-based therapy and also complicate biological studies of particular differentiation programs. Thus stem cell expansion and differentiation ex vivo are desirably controlled by ‘cocktails’ of growth factors, signaling molecules and/or genetic manipulation. Efficient and selective methods are desirable for directing the proliferation and the differentiation of stem cells, especially ES cells, to produce homogenous populations of particular cell types. This may be useful for the therapeutic use of stem cells, and to facilitate studies of the molecular mechanism of development.

Cell permeable small molecules such as dexamethasone (a glucocorticoid receptor agonist), ascorbic acid, 5-azacytidine (5-aza-C, a DNA demethylating agent) and all-trans retinoic acid have proven useful for inducing the differentiation of various stem cells. Small molecule inhibitors, such as suberoylanilide hydroxamic acid (SAHA, inhibitor of histone deacetylase-HDAC), geldanamycin (Hsp90 inhibitor), imatinib mesylate (Gleevec; kinase inhibitor) and bortezomib (proteasome inhibitor) also induce differentiation of various progenitor and transformed cells.

Among lineages of interest are those derived from mesenchymal stem cells (MSCs), which can differentiate into a variety of nonhematopoietic tissues such as osteoblasts, adipocytes and chondrocytes. Adipocyte differentiation has been shown to be favored when MSCs are plated at a high density and in adipogenic culture medium, whereas a low plating density favors osteoblastic commitment in the presence of osteogenic factors.

Adipose tissue, fat, is the largest endocrine organ in mammals and exerts a profound influence on whole body homeostasis. Adipose tissue is a major or sole source of numerous signaling molecules affecting most if not all tissues in the body, and consequently, adipose tissue plays an important regulatory role extending far beyond energy homeostasis. De novo adipocyte differentiation can be initiated during the entire lifespan of mammals by recruiting fibroblastic precursors.

Bone formation results from osteoblast lineage-specific differentiation. During osteogenesis, pluripotent mesenchymal stem cells differentiate into preosteoblasts rather than serve as progenitor cells for myocytes, adipocytes, or chondrocytes. These preosteoblasts then differentiate into mature osteoblasts that deposit the necessary components to form bone matrix and allo subsequent mineralization. Osteogenic hormones and growth factors include 1,25-(OH)₂ vitamin D₃ [1,25-(OH)₂D₃], and 17β-estradiol and bone morphogenetic proteins (BMPs). The resultant skeletal tissue supports hematopoiesis through the function of bone marrow stromal cells, which share a common progenitor with the osteogenic lineages.

Osteoporosis, a major public health burden, is associated with increased fracture risk. Fracture healing in osteoporosis is altered with reduced callus formation and impaired biomechanical properties of new formed bone leading to high risk of fixation failure. Evidence suggests that increased lipid accumulation in bone marrow correlates with decreased trabecular bone volume in osteoporotic patients. Clinical and in vitro studies support the hypothesis that plasticity exists between the adipocyte and osteoblast pathways. At the same time, obesity is becoming an increasing problem. Obesity is a prime condition predisposing to the development of different dyslipidemic conditions commonly referred to as the metabolic syndrome, and to a number of serious and common diseases such as type II diabetes, cardiovascular diseases, and certain cancers. Thus, understanding the processes that leads to differentiation of adipocytes and osteoblasts, and/or elucidating molecular pathways that control the differentiation of cells in this lineage are of considerable interest for developing new rational modalities for prevention and treatment of disorders relating to adipogenesis and osteogenesis.

Phillips et al. (2001) Biochem Biophys Res Commun 284(2):478-84; Zur Nieden et al. (2003) Differentiation 71(1):18-27; Bronson (2003) Methods Enzymol 365:241-51; Buttery et al. (2001) Tissue Eng 7(1):89-99; and Chen (2004) Bone 35(1):83-95.

Dani (2002) Methods Mol Biol 185:107-16; Dani et al. (1997) J Cell Sci 110 (Pt 11):1279-85; Phillips et al. (2003) Pharmacol Res 47:263-8; Wdziekonski et al. (2003) Methods Enzymol 365:268-77; 2003.

### SUMMARY OF THE INVENTION

Methods are provided for the in vitro differentiation of one or both of adipocytes and osteoblasts from embryonic stem cells. Embryonic stem cells are cultured to differentiate into embryoid bodies. The embryoid bodies are plated on a substrate to which they can attach. In one embodiment of the invention, the cells are differentiated into the adipocytic lineage. In this embodiment, the plated embryoid bodies are treated with all trans retinoic acid (ATRA) to initiate differentiation, then changed into an induction medium comprising insulin and triiodothyronine. Further differentiation is induced by changing into a medium comprising isobutylmethylxanthine, dexamethasone and insulin. After replating, large numbers of mature adipocytes evenly distributed among culture wells are observed, which are useful in quantitative biochemical, histochemical and molecular assays. The amount of lipid in the droplets can be stained with oil-red-O, extracted with ethanol and quantified in a colorimeter. The adipocytes are in a milieu of many other types of cells at the same developmental stage, thereby providing a physiological system for such studies.

In another embodiment of the invention, the embryoid bodies are differentiated into the osteoblastic lineage. Following plating of the embryoid bodies, the cells are treated with induction medium containing insulin and triiodothyronine, in the absence of ATRA. Further differentiation is induced by addition of ascorbic acid phosphate and β-glycerophosphate to the induction medium. The cultures are then replated, and dexamethasone optionally added to further induce osteoblast differentiation. Evenly distributed cultures containing high level of alkaline phosphatase (ALP) activity, an osteoblast marker, are produced. The increase in ALP activity is shown by the appearance of a change in cellular color which can easily be detected with a histochemical reaction and quantitated by a colorimeter. Alternatively, ALP activity can be quantitatively determined by a biochemical reaction using cell lysates.

These methods provide means to study the mechanisms of adipogenesis and osteogenesis during development; and a physiological system for drug screening to identify agents that modulate the process of adipogenesis and/or osteogenesis, particularly during early stages of development. Such methods involve combining a candidate agent with the cell population of the invention, and then determining any modulatory effect resulting from the compound. This may include examination of the cells for toxicity, metabolic change, or an effect on cell function.

These and other embodiments of the invention will be apparent from the description that follows. The compositions, methods, and techniques described in this disclosure hold considerable promise for use in diagnostic, drug screening, and therapeutic applications.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. ATRA effects on growth of MESC.

FIG. 2. Differentiation of adipocytes from EB.

FIG. 3A-3B. Differentiation of adipocytes after trypsin treatment.

FIG. 4A-4B. Hormone induced lipolysis.

### DETAILED DESCRIPTION OF THE EMBODIMENTS

Adipocytes and osteoblasts are derived from the same stem cells, and in general adipogenesis occurs at the expense of osteogenesis. Methods are provided for the in vitro differentiation of one or both of adipocytes and osteoblasts from embryonic stem cells. This enables the development of a drug screening procedure to simultaneously search for agents that modulate osteogenesis and adipogenensis.

The differentiated cells are useful for experimental evaluation, and as a source of lineage and cell specific products, including mRNA species useful in identifying genes specifically expressed in these cells, and as targets for the discovery of factors or molecules that can affect them.

### Definitions

Stem cells and cultures thereof Pluripotent stem cells are cells derived from any kind of tissue (usually embryonic tissue such as fetal or pre-fetal tissue), which stem cells have the characteristic of being capable under appropriate conditions of producing progeny of different cell types that are derivatives of all of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci USA 92:7844); marmoset stem cells (Thomson et al. (1996) Biol. Reprod. 55:254); and human embryonic germ (hEG) cells (Shamblott et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see US 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA4, TRA-I-60, TRA-1-81, and alkaline phosphatase. The globo-series glycolipid GL7, which carries the SSEA4 epitope, is formed by the addition of sialic acid to the globo-series glycolipid Gb5, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-1. Methods for proliferating hES cells in the undifferentiated form are described in WO 99/20741, WO 01/51616, and WO 03/020920.

Culture conditions of interest provide an environment permissive for differentiation, in which stem cells will proliferate, differentiate, or mature in vitro. Such conditions may also be referred to as differentiative conditions. Features of the environment include the medium in which the cells are cultured, any growth factors or differentiation-inducing factors that may be present, and a supporting structure (such as a substrate on a solid surface) if present. Differentiation may be initiated by formation of embryoid bodies (EB), or similar structures. For example, EB can result from overgrowth of a donor cell culture, or by culturing ES cells in suspension in culture vessels having a substrate with low adhesion properties.

In one embodiment of the invention, embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks.

Differentiating Cells. In the context of cell ontogeny, the adjective “differentiated”, or “differentiating” is a relative term. A “differentiated cell” is a cell that has progressed further down the developmental pathway than the cell it is being compared with. Thus, embryonic stem cells can differentiate to lineage-restricted precursor cells (such as a mesenchymal stem cell), which in turn can differentiate into other types of precursor cells further down the pathway (such as a pre-osteoblast), and then to an end-stage differentiated cell (such as an osteoblast), which plays a characteristic role in a certain tissue type, and may or may not retain the capacity to proliferate further.

Adipocyte. Adipocytes are cells present in adipose tissue, specialized in storing energy as fat. It is now well known that adipose tissue is not an inert tissue purely for the storage of energy, adipocytes secrete a large number of factors (lipids and proteins) that are termed adipokines. These adipokines can have a variety of functions in an autocrine, paracrine or endocrine fashion (see review by Kershaw and Flier, (2004) http://jcem.endojournals.org/cgi/reprint/89/6/2548). There are two types of adipose tissue (white fat and brown fat) and consequently, two types of adipocytes. White adipocytes contain large lipid vacuoles surrounded by a ring of cytoplasm. The nucleus is flattened and located on the periphery. The fat stored is in a semi-liquid state, and is composed primarily of triglycerides. White adipocytes secrete adiponectin, resistin and leptin. Brown adipocytes are polygonal in shape. Unlike white adipocyte, these cells have considerable cytoplasm with lipid droplets scattered throughout. The nucleus is round and although eccentrically located, they are not in the periphery of the cell. BAT are typically contain multiocular lipid droplet, and one characteristic of BAT is that it has a greater concentration of mitochondria, and is critically involved in thermogenesis. Detection of the lipid vesicles can be performed by, for example, staining with Oil Red O dye. In addition to the presence of lipid vacuoles, adipocytes may be characterized by the presence of the markers PPARγ, aP2, and HSL.

Osteoblast. Expression of osteoblast markers follows a clear temporal sequence. Initially, type I procollagen mRNA and collagen synthesis is induced, followed by induction of alkaline phosphatase, osteocalcin, Cbfa1, PAL, BSP, osteopontin, and the like. In addition, osteoblasts may form a mineralized extracellular matrix, which can be highly organized and contain well-banded collagen fibrils. Cellular condensations are the areas in which future bone nodule formation may be seen. The first zones of mineralization are noticed around the granules; and at more advanced stages, true mineralized bone nodules can be formed in culture.

Markers. The markers for adipocytes and osteoblasts are as described above. A number of well-known markers can be used for positive identification or selection of differentiated cells. Markers for negative selection are also of interest, particularly markers that are selectively expressed on ES cells, fibroblasts, epithelial cells, etc. Epithelial cells may be selected for as ApCAM positive. A fibroblast specific selection agent is commercially available from Miltenyi Biotec (see Fearns and Dowdle (1992) Int. J. Cancer 50:621-627 for discussion of the antigen). Markers found on ES cells suitable for negative selection include SSEA-3, SSEA-4, TRA-l-60, TRA-1-81, and alkaline phosphatase. Differentiation of hES cells in vitro typically results in the loss of these markers (if present) and increased expression of SSEA-1.

Markers for adipocytes include the presence of fat vesicles, and may include expression of adipocyte markers, e.g. PPARγ, Ap2, HSL, etc. Markers for osteoblasts include the presence of mineralized deposits, and the expression of one or more of collagen; alkaline phosphatase; osteocalcin; Cbfal; BSP; osteopontin; etc. Of particular interest are methods for calorimetric quantitation of alkaline phosphatase. An increase in ALP activity is shown by the appearance of a change in cellular color which can easily be detected with a histochemical reaction and quantitated by a calorimeter. In addition, ALP activity can be quantitatively determined by a biochemical reaction using cell lysates.

Specific Binding Member. The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). Especially useful reagents are antibodies specific for markers present on the desired cells (for positive identification or selection) and undesired cells (for negative identification or selection). Whole antibodies may be used, or fragments, e.g. Fab, F(ab′)₂, light or heavy chain fragments, etc. Such selection antibodies may be polyclonal or monoclonal and are generally commercially available or alternatively, readily produced by techniques known to those skilled in the art. Antibodies selected for use will have a low level of non-specific staining and will usually have an affinity of at least about 100 μM for the antigen.

Antibodies used for cell staining may be detectably labeled, e.g. with a fluorescent tag such as Texas Red, fluorescein, etc., or may be used with an enzymatic label, biotin staining, and the like.

### Differentiation

ES cells or cell lines as described above can be propagated continuously in culture, using culture conditions that promote proliferation without promoting differentiation, using methods known in the art. Methods of culture are described, for example, in U.S. Patent application 20030190748 (Serum free cultivation of primate embryonic stem cells); U.S. Patent application 20040023376 (Method of making embryoid bodies from primate embryonic stem cells); U.S. Patent application 20030008392 (Primate embryonic stem cells), each herein incorporated by reference. Conventionally, ES cells are cultured on a layer of feeder cells, typically fibroblasts derived from embryonic or fetal tissue, alternatively cells can be cultured on an extracellular matrix of Matrigel™ or laminin, in medium conditioned by feeder cells or medium supplemented with growth factors such as FGF and SCF (International patent publication WO 01/51616). Under the microscope, ES cells appear with high nuclear/cytoplasmic ratios, prominent nucleoli, and compact colony formation with poorly discernable cell junctions.

Embryoid bodies are harvested at an appropriate stage of development, which may be determined based on the expression of markers and phenotypic characteristics of the desired cell type e.g. at from about 1 to 4 weeks. Cultures may be empirically tested by staining for the presence of the markers of interest, by morphological determination, etc. In one embodiment of the invention, embryoid bodies are grown in a hanging drop culture, as shown in the present examples.

The embryoid bodies are plated on a substrate to which they can attach. Such substrates include plates coated with gelatin or other semi-solid coating, e.g. gelatin, agar, etc., as known in the art. The term “plates” as used herein may include Petri dishes or other tissue culture acceptable containers, e.g. 96 well plates, flasks, etc. The plating density may vary, for example from least about 1, more usually at least about 10, and not more than about 10³, more usually not more than about 10² EB per 10 cm plate, for example at a density of about 1-2 EB/cm².

Where differentiation to the adipocytic lineage is desired, the EB are treated with a suitable medium, for example Glasgow minimum essential medium, Knockout Dulbecco's minimum essential medium, and the like, and may comprise fetal bovine serum at a suitable concentration, e.g. at about 10%; and comprising all trans retinoic acid (ATRA) at a concentration of from about 10⁻⁶ M, from about 10⁻⁷ M, or from about 10⁻⁸ M, in culture for at least about 2 to 4 days, usually for about 3 days.

The medium is then changed to differentiation medium comprising insulin at a concentration of from about 10⁻⁶ M, or from about 10⁻⁷ M; and triiodothyronine at a concentration of from about 10⁻⁷ M, from about 10⁻⁸ M, or from about 10⁻⁹ M to continue differentiation. The medium is generally changed from about once a day to not less than once every three days, usually every two days. The cells are cultured for at least about 8 days, usually at least about 10 days, and may be cultured for at least about 14 days or more from the time that the cells are changed to differentiation medium.

Further stimulation of induction can be made by the addition of an induction cocktail to the differentiation medium, which results in the induction medium. The induction cocktail comprises isobutylmethylxanthine at a concentration of from about 0.1 to about 0.5 mM, dexamethasone at a concentration of from about 0.1 to 1 μM and insulin at a concentration of from about 0.1 to 1 μM. After treating the cells for at least about 5 to 8 days, cells are enzymatically removed from the culture dish, for example with trypsin/EDTA, collagenase/dispase, etc. and plated at a density of from about 10³ cells/cm² to about 10⁵ cells/cm², usually about 10⁴ cells/cm² in the differentiation medium on a substrate to which they can attach. Such substrates include plates coated with gelatin or other semi-solid coating, and comprise a suitable growth medium as described above. Cells with adipocyte characteristics appear in about two days. The number of the adipocytes and the size of lipid droplets increases over time, and reached a plateau at from about day 8 to about day 14.

In another embodiment of the invention, the embryoid bodies are differentiated into the osteoblastic lineage. Following plating of the embryoid bodies as described above, the cells are treated with differentiation medium containing insulin and triiodothyronine, in the absence of ATRA. Further differentiation is induced by addition of ascorbic acid phosphate at a concentration of at least about 0.1 to 0.3 mM and μ-glycerophosphate at a concentration of at least about 1 to 10 mM (osteoblast differentiation medium) to the differentiation medium. The cells are maintained in such culture for at least about 5 days and not more than about 14 days, usually around about 9 days.

The cells are enzymatically removed from the culture dish as described above, and plated at a density from about 10³ cells/cm² to about 10⁵ cells/cm², usually about 10⁴ cells/cm², on a substrate with osteoblast differentiation medium. Optionally, to enhance osteoblast differentiation, the medium is amended to include dexamathasone at a concentration of at least about 10⁻⁸ to 10⁻⁷ M starting at least 1 day but no more than 3 days after plating. The medium is generally changed from about once a day to not less than once every three days, usually every two days. Cells with osteoblastic characteristics are observed in at least about 7 days but usually not more than about 14 days.

The composition of differentiated cells is enriched for the desired differentiating cell type or lineage. Usually at least about 10% of the cells will comprise the desired differentiated cells, more usually at least about 15% of the aggregates, and the desired cells may be at least about 23% or more of the total cells. Further enrichment for the desired cell type may be obtained by selection for markers characteristic of the cells, e.g. by flow cytometry, magnetic bead separation, panning, etc., as known in the art.

The methods and compositions thus obtained have a variety of uses in clinical therapy, research, development, and commercial purposes. For example, the cells find use in screening assays for factors, other agents and gene expression patterns in osteogenesis and/or adipogenesis; which may include, without limitation, assays for determining factors, other agents and gene expression patterns involved differentiation of adipocytes, differentiation of osteocytes, and trans-differentiation from adipocytes to osteocytes. Areas of interest include de-differentiation of adipocytes to stem cells, with or without reentry into the differentiation process, for example to osteocytes. Another assay of interest is the induction of apoptosis in adipocytes.

Cells may be genetically altered in order to introduce genes useful in the differentiated cell, e.g. repair of a genetic defect in an individual, selectable marker, etc., or genes useful in selection against undifferentiated ES cells. Cells may also be genetically modified to enhance survival, control proliferation, and the like. Cells may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over a 8-16 h period, and then exchanged into growth medium for 1-2 days. Genetically altered cells are selected using a drug selection agent such as puromycin, G418, or blasticidin, and then recultured.

The cells of this invention can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, cardiotropic factors such as atrial natriuretic factor, cripto, and cardiac transcription regulation factors, such as GATA-4, Nkx2.5, and MEF2-C.

Many vectors useful for transferring exogenous-genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20):1 1939-44).

Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431¥437); PA317 (Miller et al. (1986) Mol. Cell. Biol. 6:2895¥2902) GRIP (Danos et al. (1988) PNAS 85:6460¥6464). Retroviruses packaged with xenotropic envelope protein, e.g. AKR env, are capable of infecting most mammalian cell types, except murine cells.

The vectors may include genes that must later be removed, e.g. using a recombinase system such as Cre/Lox, or the cells that express them destroyed, e.g. by including genes that allow selective toxicity such as herpesvirus TK, bcl-xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

The cells of this invention can be used to prepare a cDNA library relatively uncontaminated with cDNA preferentially expressed in cells from other lineages. For example, cells are collected by centrifugation at 1000 rpm for 5 min, and then mRNA is prepared from the pellet by standard techniques (Sambrook et al., supra). After reverse transcribing into cDNA, the preparation can be subtracted with cDNA from undifferentiated ES cells, other progenitor cells, or end-stage cells from the developmental pathway.

Of particular interest is the examination of gene expression in the differentiating of the invention. The expressed set of genes may be compared against other subsets of cells, against ES cells, against adult heart tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Patent No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed for the level of polypeptide of interest. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection. (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

The cells are also useful for in vitro assays and screening to detect factors that are active on differentiating cells. Of particular interest are screening assays for agents that are active on human cells. A wide variety of assays may be used for this purpose, including immunoassays for protein binding; determination of cell growth, differentiation and functional activity; production of factors; and the like.

In screening assays for biologically active agents, viruses, etc. the subject cells, usually a culture comprising the subject cells, is contacted with the agent of interest, and the effect of the agent assessed by monitoring output parameters, such as expression of markers, cell viability, and the like. The cells may be freshly isolated, cultured, genetically altered as described above, or the like. The cells may be environmentally induced variants of clonal cultures: e.g. split into independent cultures and grown under distinct conditions, for example with or without virus; in the presence or absence of other cytokines or combinations thereof. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

In addition to complex biological agents, such as viruses, candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 μl to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, NY, 2000; Freeman et al. (1999) Biotechniques 26(1):112-225; Kawamoto et al. (1999) Genome Res 9(12):1305-12; and Chen et al. (1998) Genomics 51(3):313-24, for examples.

The differentiated cells may be used for tissue reconstitution or regeneration in a human patient or other subject in need of such treatment. The cells are administered in a manner that permits them to graft or migrate to the intended tissue site and reconstitute or regenerate the functionally deficient area.

The differentiated cells may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells can be stored in a 10% DMSO, 50% FCS, 40% RPMI 1640, Glasgow MEM, Knockout DMEM medium, etc. Once thawed, the cells may be expanded by use of growth factors and/or feeder cells associated with progenitor cell proliferation and differentiation.

The cells of this invention can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types, especially endothelial cells.

For further elaboration of general techniques useful in the practice of this invention, the practitioner can refer to standard textbooks and reviews in cell biology, tissue culture, embryology, and cardiophysiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence.

Moreover, due to biological functional equivalency considerations, changes can be made in protein structure without affecting the biological action in kind or amount. All such modifications are intended to be included within the scope of the appended claims.

### EXAMPLES

### Example 1

### Adipogenesis

Mouse embryonic stem (ES) cells were used as the source of embryoid bodies (EBs). E14Tg2A cells were trypsinized and made into suspension in Glasgow Minimum essential medium containing 10% fetal bovine serum (growth medium). Cells were counted under a microscope using a haemocytometer, and were diluted to a concentration on 10⁵ cells/mi. During cell passages, 1000 U/ml of leukemia inhibitory factor (LIF) was added to prevent differentiation.

To make EBs, cell suspensions after trypsinization were made into hanging droplets (20 μl) onto the cover of a bacteriological petri dish. The bottom of the dish was filled with PBS.

20 μl droplets of cells in suspension were pipetted onto the cover of a 100 mm bacteriological petri dish. After filling the whole area of the cover (˜80 drops) with droplets, the cover was inverted to cover the bottom of the dish, which was filled with 5 ml of phosphate buffered saline (PBS). The dish was incubated at 37° C. in a 5% CO₂ incubator with saturated humidity in order to differentiate the cells into EBs.

Two days afterwards, the droplets containing EBs were collected by adding medium to the cover and equally distributed onto 2 gelatinized (precoated with 0.1% gelatin) 100 mm petri dishes. The attached EBs were treated with 10⁻⁶ M ATRA (all-trans retinoic acid) for 3 days to initiate differentiation. Medium was then changed into growth medium with 10⁻⁷ M insulin and 2×10⁻⁹ M of triiodothyronine (T₃) (induction medium) to continue adipocyte differentiation. Medium was changed every two days. At day 10, further stimulation of induction was made by the addition of an induction cocktail [0.5 mM isobutylmethylxanthine (IBMX), 0.1 μM dexamethasone (DEX) and 1 μM of insulin]. After treating the cells for 8 days, cells were removed from the dish, counted with a hemocytometer and plated onto gelatinized 6-well culture plates at 10⁴/cm². Cells with minute lipid droplets (adipocytes) appeared in two days. The number of the adipocytes and the size of droplets increased over time and reached a plateau at day 10.

Results

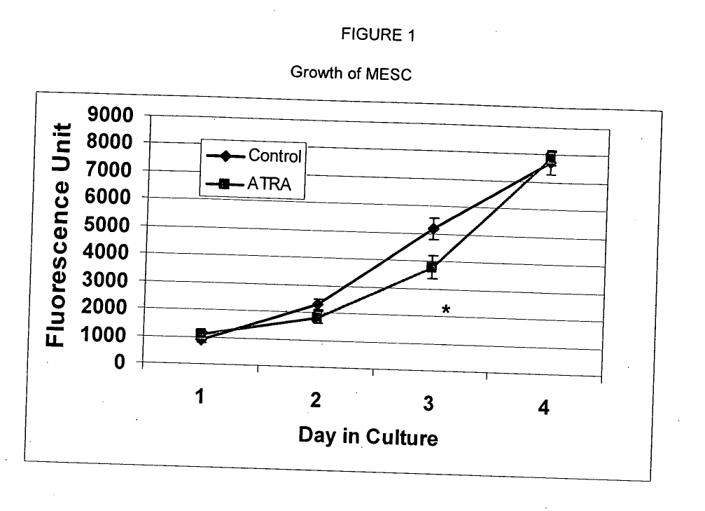

FIG. 1. ATRA effects on growth of MESC. Cells were plated at 500/well in a 96-well culture plate. Starting on day 1, they were treated with ATRA (10⁻⁶ M) for three days with replacement of fresh media each day. Control samples were treated with 0.1% DMSO (vehicle). At each indicated day, Alamar blue (10 μl/0.1 ml), an indicator for cell proliferation detection, was added to each well and the fluorescence generated was read after 4 hours.

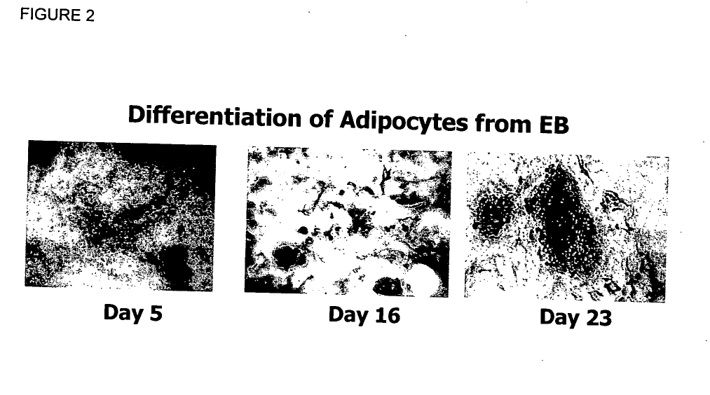

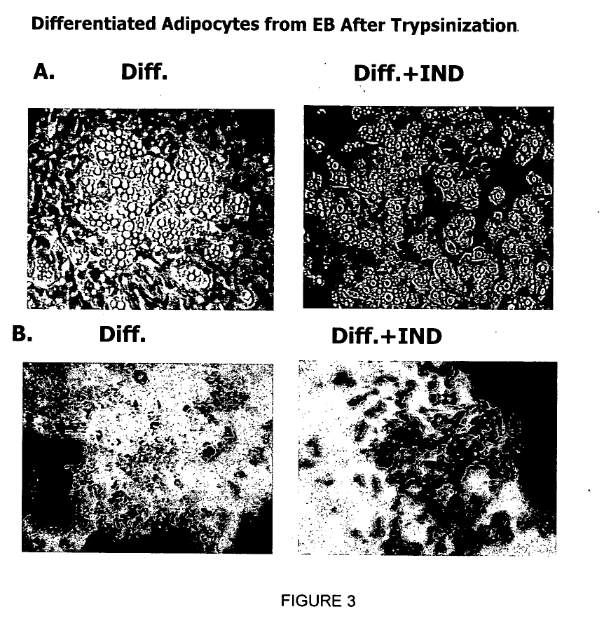

Differentiation induced by ATRA (see FIGS. 2, 3; Table 1) does not require the inhibition of cell proliferation. A significant lowering of the cell number was only seen at day 3 (p<0.05) in ATRA treatment.

FIG. 2. Differentiation of adipocytes from EBs. EBs were first treated with ATRA then cultured in differentiation medium containing insulin and T3 and left to differentiate without trypsinization. Cultures were stained with oil-red-O, alkaline phosphatase activity and hematoxylin at the indicated days starting from the beginning of the differentiation (45x). EBs show a progressive increase in the number of adipocytes with time.

FIG. 3. Differentiation of adipocytes after trypsin treatment. Cultures were differentiated as described in FIG. 2. After 11 days in the differentiation medium, EB were trypsinized and split 1:6 into gelatinized plates, then treated with induction medium containing IBMX, dexamethasone and insulin in the differentiation medium for 6 days. Photos were taken 6 days after induction ended. A. Phase contrast photos were taken at 90x. B. Bright field photos after staining with oil-red-O, alkaline phosphatase and hematoxylin. (45x). The size of lipid droplets in the adipocytes was smaller in the cells treated with induction medium. The adipocytes were more distinct with IND treatment.

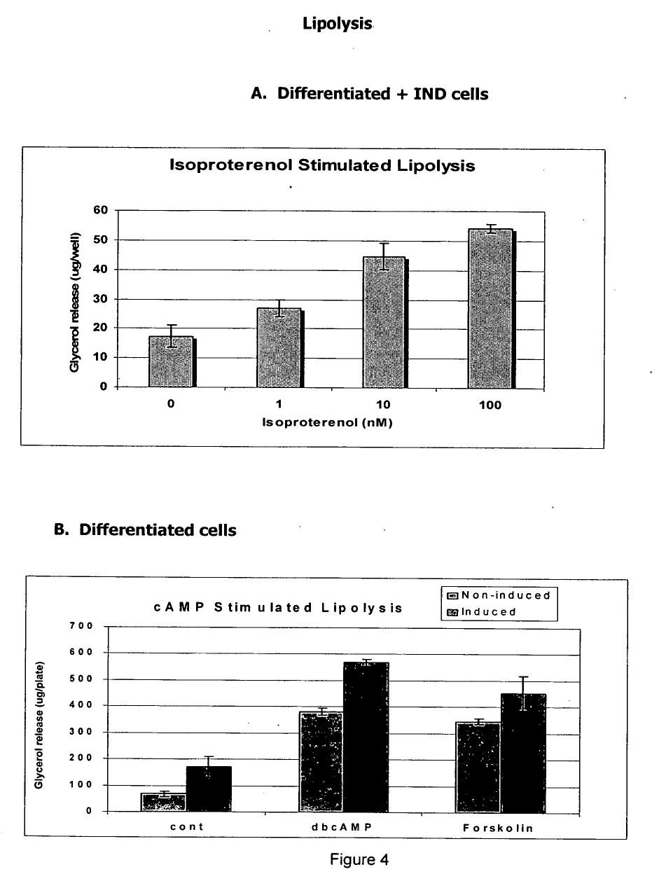

FIG. 4. Hormone induced lipolysis. Fully differentiated and induced cells were treated with increasing concentrations of isoproterenol in glucose-free DMEM containing 30 mg/ml of fatty acid free albumin and adenosine deaminase (1 U/ml) for 4 hr. The control samples were incubated with 10⁻⁵ M N-(2-phenylisopropyl)-adenosine to replace the endogenous adenosine. The amount of glycerol in the medium was measured using a calorimetric kit. Differentiated and induced cells incubated with isoproterenol in 35 mm wells. B. Differentiated cells with or without induction cultures were incubated with 10⁻³ M dibutyryl-cAMP or 4×10⁻⁵ M forskolin in 60 mm diameter culture plates. There is a dose-dependent increase of glycerol release stimulated by isoproterenol, dibutyryl-cAMP and forskolin. Induced cells showed significant increases in basal and stimulated lipolysis.

TABLE 1
Gene expression profile was characterized
by Taqman real time PCR analysis.
Analysis of Gene Expression in Differentiated MESC
Fold Change (ATRA/Control)

| | |
|---|---|
| Adipocyte Marker | Osteoblast Marker |

| | | | |
|---|---|---|---|
| ALBP | +53.2 ± 0.09 | ALP | −2.54 ± 0.03 |
| HSL | +2.39 ± 0.17 | Osteonectin | +2.02 ± 0.25 |
| PPAR-γ | +1.72 ± 0.42 | Osteocalcin | −5.28 ± 0.05 |
| | | Collagen type 1α | +6.27 ± 0.26 |

ATRA treated cultures and vehicle treated control cultures underwent the same differentiation process as described in the Methods. At the end of the culture, the ATRA treated cultures formed numerous fully differentiated adipocytes while no adipocytes appeared in the vehicle treated cultures. Total RNA was extracted, and reverse-transcribed. The relative mass of specific RNA was calculated by the comparative cycle of threshold detection method according to the manufacturer's instruction. Two independent experiments were performed using different RNA preparations from the cultures; each run of PCR was conducted in triplicate. The expression of adipocyte markers was increased while some of the osteoblast markers were decreased.

The methods described above provide a means to study the mechanisms of lipid biosynthesis and lipolysis at various stages of adipocyte differentiation. Markers at different stages of differentiation can be identified. Agents can be added at any stage of development to perturb the process. Moreover, using ES cells derived from transgenic and knock out mice reveals various gene functions.

These methods are expanded into using 96-well culture plates for high throughput drug screening for effects on adipocyte functions. For example, obesity can occur early in childhood. The number of adipocytes and/or the precursors of adipocytes at a young age help to determine the degree of adult obesity later in life. Stem cell cultures provide a large window for screening of potential drug interference during the developmental process for reduction of adipocytes early in life

### Example 2

### Osteogenesis

Mouse embryonic stem cells (ES) were used as the source of embryoid bodies. E14Tg2A cells were trypsinized and made into suspension in Glasgow Minimum essential medium containing 10% fetal bovine serum (growth medium). Cells were counted under a microscope using a haemocytometer, and were diluted to a concentration on 10⁵ cells/ml.

20 μl droplets of cells in suspension were pipetted onto the cover of a 100 mm bacteriological petri dish. After filling the whole area of the cover (˜80 drops) with droplets, the cover was inverted to cover the bottom of the dish, which was filled with 5 ml of phosphate buffered saline (PBS). The dish was incubated at 37° C. in a 5% CO₂ incubator with saturated humidity in order to differentiate the cells into embryoid bodies (EB).

Two days afterwards, the droplets containing EBs were collected by adding medium to the cover and equally distributed onto 2 gelatinized (precoated with 0.1% gelatin) 100 mm petri dishes. Without treating with ATRA, the attached EBs were incubated in the induction medium containing 10⁻⁷ M insulin and 2×10⁻⁹ M T₃ for 5 days.

After that, 0.3 mM of ascorbic acid phosphate (magnesium salt, Waco Chemicals, Dallas, Tex.) and 10 mM of β-glycerophosphate (Sigma-Aldrich Chemicals, St. Louis, Mo.) were added to the induction medium for an additional 9 days. At day 14, cultures were trypsinized to remove the cells and plated on to gelatin-coated 6-well culture plates at a density of 10⁴ cells/cm². Three days after, at day 17, DEX at 10⁻⁸ M was added to some of the wells to further induce osteoblast differentiation. Medium was replaced every 2 days throughout the culture period.

The cultures were terminated 2 weeks after at day 31 and stained for alkaline phosphatase (ALP) activity using a kit from Sigma following manufacturer's procedure (procedure No. 85). In this procedure, cells containing ALP activity, an osteoblast maker, appear in blue color. We found cultures treated with DEX had higher ALP activity showing darker blue color as compared with the ones without the treatment. The differentiation into osteoblasts was confirmed by real time reverse-transcription polymerase chain reaction (RT-PCR) analysis of mRNA in the cells showing nearly 2 fold increase in the expression of ALP and 8 fold increase in osteocalcin, another osteoblast marker for late stage osteoblast differentiation.

The compositions and procedures provided in the description can be effectively modified by those skilled in the art without departing from the spirit of the invention embodied in the claims that follow.

1. A method for in vitro differentiation of mesenchymal lineage cells from embryonic: stem cells, the method comprising:
culturing said embryonic stem cells to embryoid bodies;
plating said embryoid bodies to adhere to a semi-solid substrate;
culturing said embryoid bodies in induction medium comprising insulin and triiodothyronine;
dissociating said embryoid bodies;
replating dissociated cells to adhere to a semi-solid substrate;
culturing for a period of time sufficient for the formation of differentiated mesenchymal lineage cells.

2. The method according to claim 1, wherein said differentiated mesenchymal lineage cells are adipocytes, wherein said method further comprises:
prior to said culturing in induction medium, initiating differentiation by culturing said embryoid bodies in medium comprising all trans retinoic acid (ATRA).

3. The method according to claim 2, wherein said ATRA is present at a concentration of 10⁻⁶ M.

4. The method according to claim 2, further comprising, following said culturing in induction medium, culturing said embryoid bodies in differentiation medium comprising isobutylmethylxanthine, dexamethasone and insulin.

5. The method according to claim 4, wherein said differentiation medium comprises isobutylmethylxanthine at a concentration of about 0.5 mM, dexamethasone at a concentration of about 0.1 μM and insulin at a concentration of about 5 μg.

6. The method according to claim 1, wherein said semi-solid substrate comprises a gelatin coating.

7. The method according to claim 1, wherein said differentiated mesenchymal lineage cells are osteoblasts.

8. The method according to claim 7, further comprising, following said culturing in induction medium, culturing said embryoid bodies in differentiation medium comprising ascorbic acid phosphate and β-glycerophosphate.

9. The method according to claim 8, wherein said differentiation medium comprises ascorbic acid phosphate at a concentration of about 0.3 mM and β-glycerophosphate at a concentration of about 10 mM.

10. The method according to claim 9, wherein said cells are cultured in the presence of dexamethasone following dissociation and replating.

11. The method according to claim 10, wherein said dexamethasone is present at a concentration of about 10⁻⁸ M.

12. The method according to claim 10, wherein the presence of osteoblasts is detected by calorimetric quantitation of alkaline phosphatase.

13. An in vitro cell culture produced by the method according to claim 5, and comprising adipocytes.

14. An in vitro cell culture produced by the method according to claim 11, and comprising osteoblasts.

15. The in vitro culture of claims 13, wherein said cells are human cells.

16. The in vitro culture of claims 13, wherein said cells are mouse cells.

17. A method of screening for genetic sequences specifically expressed in differentiating cells of the mesenchymal lineage, the method comprising:
isolating RNA from a cell population according to any one of claims 13,
generating a probe from said RNA,
screening a population of nucleic acids for hybridization to said probe.

18. A method of screening for agents that affect the viability, growth, metabolic function or differentiation of differentiating cells of the mesenchymal lineage, the method comprising:
contacting a cell population according to any one of claims 13, and
determining the effect of said agent on the viability, growth, metabolic function or differentiation of said cells.
